(19) United States
(12) Patent Application Publication
(10) Pub. No.: US 2004/0127803 A1
(43) Pub. Date: Jul. 1, 2004

(54) Arrangement and method for recording signals of biological origin

(76) Inventors: Sebastian Berkes, Ilmenau (DE); Galina Ivanova, Ilmenau (DE); Falk Schlegemilch, Ilmenau (DE); Klaus Schellhorn, Ilmenau (DE); Peter Husar, Ilmenau (DE); Guenter Henning, Ilmenau (DE)

Correspondence Address: REED SMITH, LLP, ATTN: PATENT RECORDS DEPARTMENT, 599 LEXINGTON AVENUE, 29TH FLOOR, NEW YORK, NY, 10022-7650, US

(21) Appl. No.: 10/474,049

(22) PCT Filed: Apr. 4, 2002
(86) PCT No.: PCT/DE02/01320

(30) Foreign Application Priority Data
Apr. 5, 2001 (DE) 101-17-155.2

(51) Int. Cl. A61B 5/04

(52) U.S. Cl. 600/509; 600/544; 600/546

(57) ABSTRACT

The invention relates to the multi-channel recording of signals of various biological origins in the frequency range of 0 to several kilohertz, preparation of the reference potential for the differential amplifier on each channel, from the determined data from the analogue to digital converter and predominantly, though not exclusively, all areas of medicine in which biosignals are used.

The invention is directed to an arrangement and a method for the acquisition of signals of biological origin. This method and the arrangement are applied primarily, but not exclusively, to all areas of medicine in which biosignals are used.

Biological signals supply information about the function of organs within an organism. The evaluation of biosignals is used as a diagnostic tool in medicine (EKG, EEG, EMG, EOG, ERG, PPT, respiration, MKG, MEG). Aside from adequate signal processing and feature extraction, a precondition for the quality of diagnosis is a signal acquisition that is free from artifacts and interference.

In this connection, the followings points must be considered: After conversion in the range of nanovolts to millivolts, the signal levels lie within a frequency band from zero to several kilohertz; strong interference signals occur in the frequency band that is used; the signal sources, e.g., of electrophysiological origin, to be examined have high impedance; the physical characteristics, e.g., of the electrodes, change over time (e.g., through variation in the inter-electrode impedance, electrode voltage, offset potential, contact pressure condition, movement artifacts).

Signal acquisition systems known in the art partially overcome these problems through a careful selection of derivation methodology and appropriate amplifier technology. High-quality commercial polygraphy systems for recording biosignals of different physiological origins are very cost-intensive and are usually provided only for stationary use.

The present procedure using electrodes is described in the following as an example for the acquisition of signals of biological origin.

The biological signal is tapped from the tissue under examination by means of electrodes and is fed via electrode cable to a differential amplifier whose artificial or synthetic reference potential can be generated in analog from the sum of all connected electrodes (common average). This measurement arrangement is simple but very sensitive to interference. For this reason, measurements—e.g., of the electroencephalogram (EEG)—can be carried out only in a low-interference environment or after laborious measures to eliminate interference (Faraday cage, local shielding). The construction of these acquisition systems is complex because every channel has its own analog preprocessing stage. This increases susceptibility to interference, constructional size and energy consumption and impedes parameter matching of the channels. The DC component of the biosignals is suppressed by an analog high-pass filter.

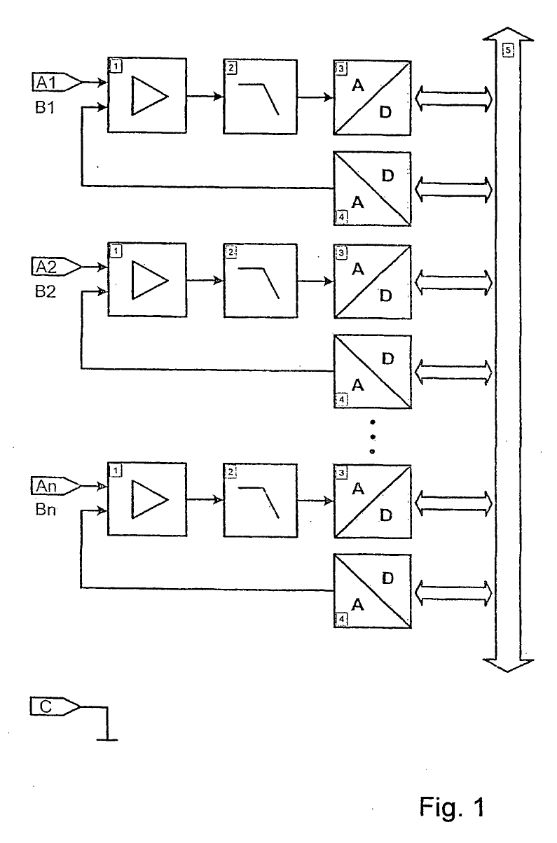

Exacting methods for biosignal acquisition and evaluation require highly efficient biosignal amplifiers which can also acquire signal components in the low-frequency range up to DC voltage without distortion. This can be realized when an analog high-pass filter is done away with entirely and the total filter functionality, with the exception of the anti-aliasing filter, is shifted to the digital plane. All of the differential signals generated and measured in the system presented (FIG. 1) refer to a common ground potential C which can be derived from the measured object. Each channel contains a differential amplifier 1, an anti-aliasing filter 2, an analog-to-digital converter 3, and a digital-to-analog converter 4 and is decoupled from the other channels. In all channels n, the difference between input signal Aₙ and a reference potential Bₙ, both of which refer to the ground potential C, are amplified, filtered and digitized. The anti-aliasing filter 2 connected in the channel path serves to limit the frequency range and, accordingly, to adhere to the sampling theorem during subsequent quantization in the analog-to-digital converter 3. The data are provided on a data and control bus 5 and are further processed either in the acquisition system itself or in another system after data transfer. The reference potential Bₙ of every differential amplifier 1 is determined from the data of the respective analog-to-digital converter 3 and is sent back to the complementary input via a digital-to-analog converter 4. In this way, possible overloading of the differential amplifier 1 is prevented without losing the information about the DC component.

In order to acquire the signal of biological origin, the differential signal between two channels, e.g., A₁ and A₂, is formed by digital subtraction either in the acquisition system itself or in another system after data transfer. This makes it possible to designate any channel as reference channel in order to realize unipolar derivations. It is also conceivable to define a plurality of independent reference channels, for example, for biosignals of different origin.

The adjusted gains for each channel n should be equal in order to obtain sufficient suppression of the influence of the common mode signal on the results. The gain can be set in such a way that the amplitude of virtually all biosignals can be acquired without losing information due to overload, quantization or system noise.

This arrangement has the following substantial advantages compared to conventional solutions:

No analog high-pass filtering is necessary, so that precision components and time-consuming parameter matching thereof is done away with.

Signal acquisition in the low-frequency range to DC voltage is possible.

Data processing is carried out completely digitally.

Since the derivation is carried out at ground potential, the measurement data are unipolar after digital subtraction.

Starting from the unipolar measurement data mentioned above, any reference channels can be generated independent from hardware.

A simultaneous acquisition of biosignals of different origin is possible with different gain factors and sampling rates.

The modular hardware concept of the channels and the common digital interface enable any cascading.

The data are not acquired by time multiplexing as in conventional systems, but can be scanned simultaneously or completely independent from one another due to the modular structure.

The digital interface enables very efficient galvanic separation of the measuring arrangement from the evaluating equipment, so that costly analog isolation amplifiers for ensuring safety during medical use are eliminated without jeopardizing the safety of the measured subject (patient).

Compared to the conventional solutions, the proposed solution is characterized by compact size and low energy requirement.

Analog-to-digital conversion can be carried out very close to the signal source due to the small constructional size. Interference is accordingly reduced because analog signal paths are very short and interference that is coupled in by induction via conductor loops in the analog part of the hardware is prevented. Conventional amplifiers can not separate inductively coupled-in interference from the useful signal, since they are present as differential input voltage or current and are amplified by the useful signal.

Abstract

Multiple-channel acquisition of signals of various biological origin in the frequency range of 0 to several kilohertz. Preparation of the reference potential of the differential amplifier at each channel from the determined data from the analog-to-digital converter. Primarily, but not exclusively, all areas of medicine in which biosignals are used.

### Reference Numbers

1 differential amplifier

2 anti-aliasing filter

3 analog-to-digital converter

4 digital-to-analog converter

5 data and control bus

Abbreviations

| | |
|---|---|
| EKG | electrocardiogram |
| EEG | electroencephalogram |
| EMG | electromyogram |
| EOG | electrooculogram |
| ERG | electroretinogram |
| PPT | photoplethysmography |
| MKG | magnetocardiogram |
| MEG | magnetoencephalogram |

1. Method for the acquisition of signals of biological origin, characterized in that the signals coming from a biological source that are converted into an electrical quantity are amplified and quantized, each channel has its own digitally controlled reference potential, and a common ground potential derived from the measured object is used.

2. Method according to claim 1, characterized in that the signals can be applied digitally to one or more reference channels.

3. Method according to one of claims 1 or 2, characterized in that the simultaneous acquisition of multiple-channel biological signals of the same and/or different origin is possible.

4. Arrangement for the acquisition of signals of biological origin, characterized in that the biosignals that are converted into an electrical quantity are amplified by a differential amplifier 1 and are digitized by an analog-to-digital converter 3 following an anti-aliasing filter 2, and the reference potential Bₙ obtained from the data of the analog-to-digital converter 3 is made available at the complementary input of the differential amplifier 1 by a digital-to-analog converter 4.
